(19) United States
(12) Patent Application Publication
(10) Pub. No.: US 2025/0303162 A1
(43) Pub. Date: Oct. 2, 2025

(54) SYSTEMS AND METHODS FOR IDENTIFYING STIMULUS FREQUENCIES TO USE DURING A LEAD INSERTION PROCEDURE

(71) Applicant: Advanced Bionics LLC, Valencia, CA (US)

(72) Inventors: Kanthaiah Koka, Valencia, CA (US); Anthony J. Spahr, Newhall, CA (US)

(21) Appl. No.: 18/622,075

(22) Filed: Mar. 29, 2024

(51) Int. Cl. A61N 1/36; A61N 1/05

(52) U.S. Cl. CPC A61N 1/36039; A61N 1/0541

(57) ABSTRACT

An exemplary system comprises memory that stores instructions and a processor communicatively coupled to the memory and configured to execute the instructions to perform a process. The process may comprise obtaining a pre-operative audiogram of a recipient, the pre-operative audiogram generated prior to a lead insertion procedure during which an electrode lead having a plurality of electrodes is inserted into a cochlea of the recipient, obtaining one or more attributes associated with the lead insertion procedure, and determining, based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

### BACKGROUND INFORMATION

Cochlear implant systems are used to provide, restore, and/or improve the sense of hearing to recipients with severe or profound hearing loss. Conventional cochlear implant systems include various components configured to be implanted within a recipient. For example, an electrode lead may be inserted into a cochlea of the recipient and stimulation current may be applied by electrodes on the electrode lead as directed by a cochlear implant that is also surgically implanted within the recipient.

The insertion of an electrode lead into the cochlea of the recipient is performed by way of a delicate surgical procedure that can result in the electrode lead being mispositioned and/or causing trauma to the cochlea of the patient. In view of this, it is important to carefully monitor the electrode lead during insertion into the cochlea, which monitoring may include applying stimulation (e.g., acoustic stimulation) to elicit evoked responses within the recipient. Such evoked responses may be used to determine whether, for example, trauma (e.g., a translocation of the electrode lead from the scala tympani to the scala vestibuli) has occurred during the lead insertion procedure. Typically, stimulation having a predetermined stimulation frequency (e.g., 500 Hz) is used to elicit the evoked responses based on residual hearing presence of recipients. However, the predetermined single frequency may not be optimal in situations where the electrode lead does not reach a location within the cochlea associated with the predetermined frequency and may require more than single frequency to activate in the cochlea. Moreover, because different recipients of cochlear implant systems have different hearing capabilities, certain stimulation frequencies used during a lead insertion procedure may be suitable for some recipients but not other recipients.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system.

FIG. 2 shows an exemplary configuration of the cochlear implant system of FIG. 1.

FIG. 3 shows another exemplary configuration of the cochlear implant system of FIG. 1.

FIG. 4 shows an exemplary lead insertion management system according to principles described herein.

FIG. 5 shows an exemplary flow diagram depicting operations that may be performed according to principles described herein.

FIGS. 6-8 show exemplary pre-operative audiograms and stimulation parameters that may be implemented according to principles described herein.

FIG. 9 shows an exemplary method according to principles described herein.

FIG. 10 shows an exemplary computing device that may be implemented according to principles described herein.

### DETAILED DESCRIPTION

Systems and methods for identifying stimulus frequencies to use during a lead insertion procedure are described herein. An exemplary system comprises memory that stores instructions and a processor communicatively coupled to the memory and configured to execute the instructions to perform a process. The process may comprise obtaining a pre-operative audiogram of a recipient, the pre-operative audiogram generated prior to a lead insertion procedure during which an electrode lead having a plurality of electrodes is inserted into a cochlea of the recipient, obtaining one or more attributes associated with the lead insertion procedure, and determining, based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

The systems and methods described herein may provide various benefits to cochlear implant recipients, as well as others involved with managing cochlear implant systems. For example, systems and methods such as those described herein may facilitate optimizing which stimulation frequencies may be used during a lead insertion procedure based on a recipient's individual hearing capability and various other factors. As a result, systems and methods such as those described herein include a recipient specific approach that may facilitate more effectively monitoring whether the electrode lead is positioned properly (e.g., at the proper depth, has proper cochlea wall contact, etc.) or is positioned improperly (e.g., not adequately within the cochlea, has translocated the basilar membrane, etc.). In addition, systems and methods such as those described herein may facilitate providing useful feedback (e.g., by way of user interfaces, lights, sounds, etc.) during a lead insertion procedure that may provide a surgeon or other user performing the lead insertion procedure with information and perspective into the intricate lead insertion procedure, thereby allowing for a translocated electrode lead to be corrected (e.g., withdrawn and reinserted without scalar translocation) or for trauma to otherwise be mitigated to facilitate a successful outcome of the lead insertion procedure.

Various embodiments will now be described in more detail with reference to the figures. The disclosed systems and methods may provide one or more of the benefits mentioned above and/or various additional and/or alternative benefits that will be made apparent herein.

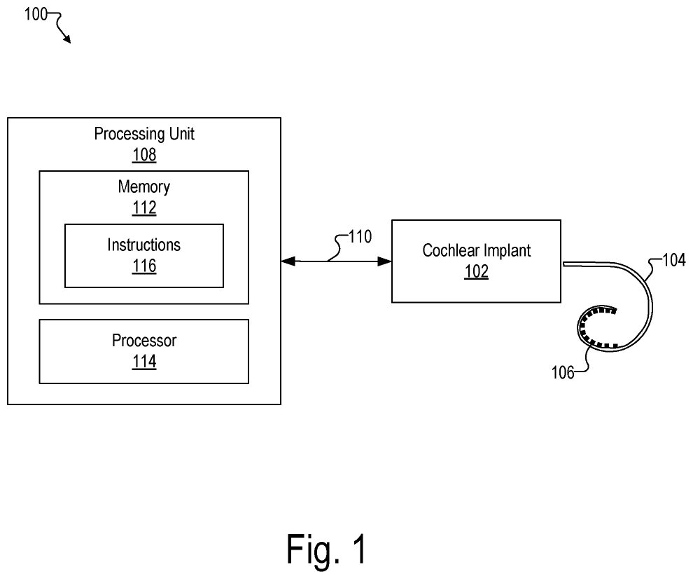

FIG. 1 illustrates an exemplary cochlear implant system 100 configured to be used by a recipient. As shown, cochlear implant system 100 includes a cochlear implant 102, an electrode lead 104 physically coupled to cochlear implant 102 and having an array of electrodes 106, and a processing unit 108 configured to be communicatively coupled to cochlear implant 102 by way of a communication link 110.

The cochlear implant system 100 shown in FIG. 1 is unilateral (i.e., associated with only one ear of the recipient). Alternatively, a bilateral configuration of cochlear implant system 100 may include separate cochlear implants and electrode leads for each ear of the recipient. In the bilateral configuration, processing unit 108 may be implemented by a single processing unit configured to interface with both cochlear implants or by two separate processing units each configured to interface with a different one of the cochlear implants.

Cochlear implant 102 may be implemented by any suitable type of implantable stimulator. For example, cochlear implant 102 may be implemented by an implantable cochlear stimulator. Additionally or alternatively, cochlear implant 102 may be implemented by a brainstem implant and/or any other type of device that may be implanted within the recipient and configured to apply electrical stimulation to one or more stimulation sites located along an auditory pathway of the recipient.

In some examples, cochlear implant 102 may be configured to generate electrical stimulation representative of an audio signal processed by processing unit 108 in accordance with one or more stimulation parameters transmitted to cochlear implant 102 by processing unit 108. Cochlear implant 102 may be further configured to apply the electrical stimulation to one or more stimulation sites (e.g., one or more intracochlear locations) within the recipient by way of one or more electrodes 106 on electrode lead 104. In some examples, cochlear implant 102 may include a plurality of independent current sources each associated with a channel defined by one or more of electrodes 106. In this manner, different stimulation current levels may be applied to multiple stimulation sites simultaneously by way of multiple electrodes 106.

Cochlear implant 102 may additionally or alternatively be configured to generate, store, and/or transmit data. For example, cochlear implant may use one or more electrodes 106 to record one or more signals (e.g., one or more voltages, impedances, evoked responses within the recipient, and/or other measurements) and transmit, by way of communication link 110, data representative of the one or more signals to processing unit 108. In some examples, this data is referred to as back telemetry data.

Electrode lead 104 may be implemented in any suitable manner. For example, a distal portion of electrode lead 104 may be pre-curved such that electrode lead 104 conforms with the helical shape of the cochlea after being implanted. Electrode lead 104 may alternatively be naturally straight or of any other suitable configuration.

In some examples, electrode lead 104 includes a plurality of wires (e.g., within an outer sheath) that conductively couple electrodes 106 to one or more current sources within cochlear implant 102. For example, if there are n electrodes 106 on electrode lead 104 and n current sources within cochlear implant 102, there may be n separate wires within electrode lead 104 that are configured to conductively connect each electrode 106 to a different one of the n current sources. Exemplary values for n are 8, 12, 16, or any other suitable number.

Electrodes 106 are located on at least a distal portion of electrode lead 104. In this configuration, after the distal portion of electrode lead 104 is inserted into the cochlea, electrical stimulation may be applied by way of one or more of electrodes 106 to one or more intracochlear locations. One or more other electrodes (e.g., including a ground electrode, not explicitly shown) may also be disposed on other parts of electrode lead 104 (e.g., on a proximal portion of electrode lead 104) to, for example, provide a current return path for stimulation current applied by electrodes 106 and to remain external to the cochlea after the distal portion of electrode lead 104 is inserted into the cochlea. Additionally or alternatively, a housing of cochlear implant 102 may serve as a ground electrode for stimulation current applied by electrodes 106. In certain examples, electrode lead 104 may alternatively be referred to as an electrode array.

Processing unit 108 may be configured to interface with (e.g., control and/or receive data from) cochlear implant 102. For example, processing unit 108 may transmit commands (e.g., stimulation parameters and/or other types of operating parameters in the form of data words included in a forward telemetry sequence) to cochlear implant 102 by way of communication link 110. Processing unit 108 may additionally or alternatively provide operating power to cochlear implant 102 by transmitting one or more power signals to cochlear implant 102 by way of communication link 110. Processing unit 108 may additionally or alternatively receive data from cochlear implant 102 by way of communication link 110. Communication link 110 may be implemented by any suitable number of wired and/or wireless bidirectional and/or unidirectional links.

As shown, processing unit 108 includes a memory 112 and a processor 114 configured to be selectively and communicatively coupled to one another. In some examples, memory 112 and processor 114 may be distributed between multiple devices and/or multiple locations as may serve a particular implementation.

Memory 112 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 112 may maintain (e.g., store) executable data used by processor 114 to perform one or more of the operations described herein. For example, memory 112 may store instructions 116 that may be executed by processor 114 to perform any of the operations described herein. Instructions 116 may be implemented by any suitable application, program (e.g., sound processing program), software, code, and/or other executable data instance. Memory 112 may also maintain any data received, generated, managed, used, and/or transmitted by processor 114.

Processor 114 may be configured to perform (e.g., execute instructions 116 stored in memory 112 to perform) various operations with respect to cochlear implant 102.

To illustrate, processor 114 may be configured to control an operation of cochlear implant 102. For example, processor 114 may receive an audio signal (e.g., by way of a microphone communicatively coupled to processing unit 108, a wireless interface (e.g., a Bluetooth interface), and/or a wired interface (e.g., an auxiliary input port)). Processor 114 may process the audio signal in accordance with a sound processing program (e.g., a sound processing program stored in memory 112) to generate appropriate stimulation parameters. Processor 114 may then transmit the stimulation parameters to cochlear implant 102 to direct cochlear implant 102 to apply electrical stimulation representative of the audio signal to the recipient.

In some implementations, processor 114 may also be configured to apply acoustic stimulation to the recipient. For example, a receiver (also referred to as a loudspeaker) may be optionally coupled to processing unit 108. In this configuration, processor 114 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 114 is configured to both deliver acoustic stimulation to the recipient and direct cochlear implant 102 to apply electrical stimulation to the recipient, cochlear implant system 100 may be referred to as a bimodal hearing system and/or any other suitable term.

Processor 114 may be additionally or alternatively configured to receive and process data generated by cochlear implant 102. For example, processor 114 may receive data representative of a signal recorded by cochlear implant 102 using one or more electrodes 106 and, based on the data, adjust one or more operating parameters of processing unit 108. Additionally or alternatively, processor 114 may use the data to perform one or more diagnostic operations with respect to cochlear implant 102 and/or the recipient.

Other operations may be performed by processor 114 as may serve a particular implementation. In the description provided herein, any references to operations performed by processing unit 108 and/or any implementation thereof may be understood to be performed by processor 114 based on instructions 116 stored in memory 112.

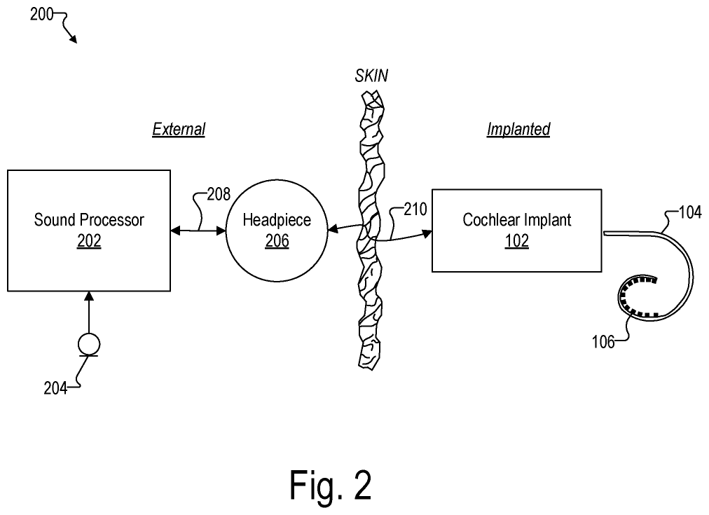

Processing unit 108 may be implemented by one or more devices configured to interface with cochlear implant 102. To illustrate, FIG. 2 shows an exemplary configuration 200 of cochlear implant system 100 in which processing unit 108 is implemented by a sound processor 202 configured to be located external to the recipient. In configuration 200, sound processor 202 is communicatively coupled to a microphone 204 and to a headpiece 206 that are both configured to be located external to the recipient.

Sound processor 202 may be implemented by any suitable device that may be worn or carried by the recipient. For example, sound processor 202 may be implemented by a behind-the-ear (“BTE”) unit configured to be worn behind and/or on top of an ear of the recipient. Additionally or alternatively, sound processor 202 may be implemented by an off-the-ear unit (also referred to as a body worn device) configured to be worn or carried by the recipient away from the ear. Additionally or alternatively, at least a portion of sound processor 202 is implemented by circuitry within headpiece 206.

Microphone 204 is configured to detect one or more audio signals (e.g., that include speech and/or any other type of sound) in an environment of the recipient. Microphone 204 may be implemented in any suitable manner. For example, microphone 204 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 202. Additionally or alternatively, microphone 204 may be implemented by one or more microphones in or on headpiece 206, one or more microphones in or on a housing of sound processor 202, one or more beam-forming microphones, and/or any other suitable microphone as may serve a particular implementation.

Headpiece 206 may be selectively and communicatively coupled to sound processor 202 by way of a communication link 208 (e.g., a cable or any other suitable wired or wireless communication link), which may be implemented in any suitable manner. Headpiece 206 may include an external antenna (e.g., a coil and/or one or more wireless communication components) configured to facilitate selective wireless coupling of sound processor 202 to cochlear implant 102. Headpiece 206 may additionally or alternatively be used to selectively and wirelessly couple any other external device to cochlear implant 102. To this end, headpiece 206 may be configured to be affixed to the recipient's head and positioned such that the external antenna housed within headpiece 206 is communicatively coupled to a corresponding implantable antenna (which may also be implemented by a coil and/or one or more wireless communication components) included within or otherwise connected to cochlear implant 102. In this manner, stimulation parameters and/or power signals may be wirelessly and transcutaneously transmitted between sound processor 202 and cochlear implant 102 by way of a wireless communication link 210.

In configuration 200, sound processor 202 may receive an audio signal detected by microphone 204 by receiving a signal (e.g., an electrical signal) representative of the audio signal from microphone 204. Sound processor 202 may additionally or alternatively receive the audio signal by way of any other suitable interface as described herein. Sound processor 202 may process the audio signal in any of the ways described herein and transmit, by way of headpiece 206, stimulation parameters to cochlear implant 102 to direct cochlear implant 102 to apply electrical stimulation representative of the audio signal to the recipient.

In an alternative configuration, sound processor 202 may be implanted within the recipient instead of being located external to the recipient. In this alternative configuration, which may be referred to as a fully implantable configuration of cochlear implant system 100, sound processor 202 and cochlear implant 102 may be combined into a single device or implemented as separate devices configured to communicate one with another by way of a wired and/or wireless communication link. In a fully implantable implementation of cochlear implant system 100, headpiece 206 may not be included and microphone 204 may be implemented by one or more microphones implanted within the recipient, located within an ear canal of the recipient, and/or external to the recipient.

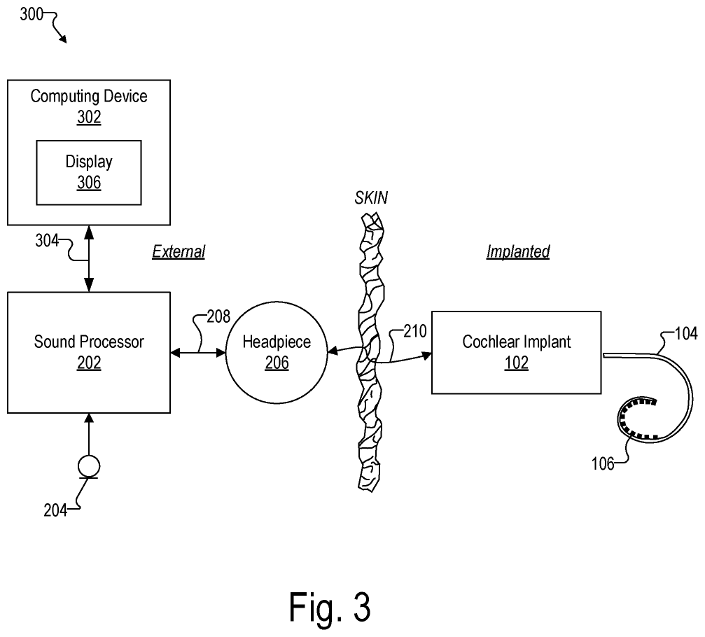

FIG. 3 shows an exemplary configuration 300 of cochlear implant system 100 in which processing unit 108 is implemented by a combination of sound processor 202 and a computing device 302 configured to communicatively couple to sound processor 202 by way of a communication link 304, which may be implemented by any suitable wired or wireless communication link.

Computing device 302 may be implemented by any suitable combination of hardware and software. To illustrate, computing device 302 may be implemented by a mobile device (e.g., a mobile phone, a laptop, a tablet computer, etc.), a desktop computer, and/or any other suitable computing device as may serve a particular implementation. As an example, computing device 302 may be implemented by a mobile device configured to execute an application (e.g., a “mobile app”) that may be used by a user (e.g., the recipient, a clinician, and/or any other user) to control one or more settings of sound processor 202 and/or cochlear implant 102 and/or perform one or more operations (e.g., diagnostic operations) with respect to data generated by sound processor 202 and/or cochlear implant 102.

In some examples, computing device 302 may be configured to control an operation of cochlear implant 102 by transmitting one or more commands to cochlear implant 102 by way of sound processor 202. Likewise, computing device 302 may be configured to receive data generated by cochlear implant 102 by way of sound processor 202. Alternatively, computing device 302 may interface with (e.g., control and/or receive data from) cochlear implant 102 directly by way of a wireless communication link between computing device 302 and cochlear implant 102. In some implementations in which computing device 302 interfaces directly with cochlear implant 102, sound processor 202 may or may not be included in cochlear implant system 100.

Computing device 302 is shown as having an integrated display 306. Display 306 may be implemented by a display screen, for example, and may be configured to display content generated by computing device 302. Additionally or alternatively, computing device 302 may be communicatively coupled to an external display device (not shown) configured to display the content generated by computing device 302.

In some examples, computing device 302 represents a fitting device configured to be selectively used (e.g., by a clinician) to fit sound processor 202 and/or cochlear implant 102 to the recipient. In these examples, computing device 302 may be configured to execute a fitting program configured to set one or more operating parameters of sound processor 202 and/or cochlear implant 102 to values that are optimized for the recipient. As such, in these examples, computing device 302 may not be considered to be part of cochlear implant system 100. Instead, computing device 302 may be considered to be separate from cochlear implant system 100 such that computing device 302 may be selectively coupled to cochlear implant system 100 when it is desired to fit sound processor 202 and/or cochlear implant 102 to the recipient.

During a lead insertion procedure, one or more of electrodes 106 may be used to record evoked responses elicited within a recipient. Such evoked responses may be elicited by stimulation (e.g., acoustic stimulation) of the recipient and may facilitate monitoring an insertion condition associated with electrode lead 104. For example, such evoked responses may provide information indicating trauma to the cochlea, a position of electrode lead 104 within the cochlea, and/or any other suitable information. Exemplary evoked responses include, but are not limited to, an electrocochleographic (ECochG) potential (e.g., a cochlear microphonic potential, a compound action potential such as an auditory nerve response, a summating potential, etc.), a brainstem response, a stapedius reflex, and/or any other type of neural or physiological response that may occur within a recipient in response to application of acoustic stimulation to the recipient. Evoked responses may originate from neural tissues, hair cell to neural synapses, inner or outer hair cells, and/or other sources.

The stimulation used to elicit such evoked responses may have any suitable stimulation frequency or combinations of stimulation frequencies as may serve a particular implementation. For example, the stimulation provided to a recipient during a lead insertion procedure may have a first stimulation frequency, a second stimulation frequency, a third stimulation frequency, and a fourth stimulation frequency. In such examples, stimulation having the first stimulation frequency, the second stimulation frequency, the third stimulation frequency, and the fourth stimulation frequency may be concurrently provided to the recipient during a lead insertion procedure. Although certain stimulation frequencies may be effective for some recipients of cochlear implants, other recipients may not be receptive to those particular stimulation frequencies or combinations of stimulation frequencies based on their hearing capability. For example, a first recipient of a cochlear implant system may have a hearing capability that is receptive to each of the first, second, third, and fourth stimulation frequencies. However, a second recipient may have a hearing capability that is only receptive to the first stimulation frequency and the second stimulation frequency. In view of this, stimulation having each of the first, second, third, and fourth stimulation frequencies may not be optimized for the second recipient. Accordingly, the systems and methods described herein take into account the individual hearing capabilities of cochlear implant recipients when identifying which stimulation frequencies to use during a lead insertion procedure to facilitate successful outcomes of the lead insertion procedures.

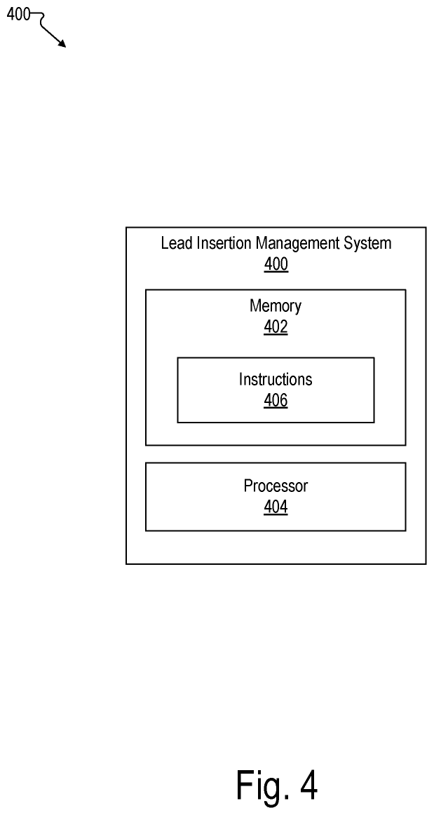

To that end, FIG. 4 shows an exemplary lead insertion management system 400 (“system 400”) that may be implemented according to principles described herein to facilitate identifying which stimulation frequencies and/or other parameters (e.g., stimulation levels) to use during a lead insertion procedure. As shown, system 400 may include, without limitation, a memory 402 and a processor 404 selectively and communicatively coupled to one another. Memory 402 and processor 404 may each include or be implemented by hardware and/or software components (e.g., processors, memories, communication interfaces, instructions stored in memory for execution by the processors, etc.). In some examples, memory 402 and/or processor 404 may be implemented by any suitable computing device. In other examples, memory 402 and/or processor 404 may be distributed between multiple devices and/or multiple locations as may serve a particular implementation. Illustrative implementations of system 400 are described herein.

Memory 402 may maintain (e.g., store) executable data used by processor 404 to perform any of the operations described herein. For example, memory 402 may store instructions 406 that may be executed by processor 404 to perform any of the operations described herein. Instructions 406 may be implemented by any suitable application, software, code, and/or other executable data instance.

Memory 402 may also maintain any data received, generated, managed, used, and/or transmitted by processor 404. Memory 402 may store any other suitable data as may serve a particular implementation. For example, memory 402 may store data associated with pre-operative audiograms, stimulation frequency information, evoked response information, predicted insertion depth information, statistical information (e.g., historical information regarding past lead insertion procedures performed on other recipients), stimulation level information, system capability information, notification information, graphical user interface content, and/or any other suitable data.

Processor 404 may be configured to perform (e.g., execute instructions 406 stored in memory 402 to perform) various processing operations associated with identifying stimulation frequencies to use during a lead insertion procedure. For example, processor 404 may perform one or more operations described herein to determine, based on a pre-operative audiogram and one or more attributes associated with a lead insertion procedure, one or more stimulation frequencies for stimulation that is to be used during the lead insertion procedure to elicit evoked responses within the recipient. These and other operations that may be performed by processor 404 are described herein.

System 400 may be implemented in any suitable manner. For example, system 400 may be implemented by any suitable computing device (e.g., a desktop computer, a laptop computer, a cloud computing device, etc.) that may be configured to access and process scan images according to operations such as those described herein.

In some examples, system 400 may be implemented by a computing device that represents a fitting device configured to be selectively used (e.g., by a clinician) to fit sound processor 202 and/or cochlear implant 102 to the recipient. In these examples, the computing device may be configured to execute a fitting program configured to set one or more operating parameters of sound processor 202 and/or cochlear implant 102 to values that are optimized for the recipient. As such, in these examples, the computing device may be considered to be separate from cochlear implant system 100 such that the computing device may be selectively coupled to cochlear implant system 100 when it is desired to fit sound processor 202 and/or cochlear implant 102 to the recipient.

In certain examples, the methods described herein may be performed automatically by system 400. As used herein, the expression “automatically” means that an operation (e.g., an operation determining which stimulation frequencies, stimulation levels, etc. to use) or series of operations are performed without requiring further input from a user. For example, system 400 may automatically perform any of the operations described herein without requiring further input from a user.

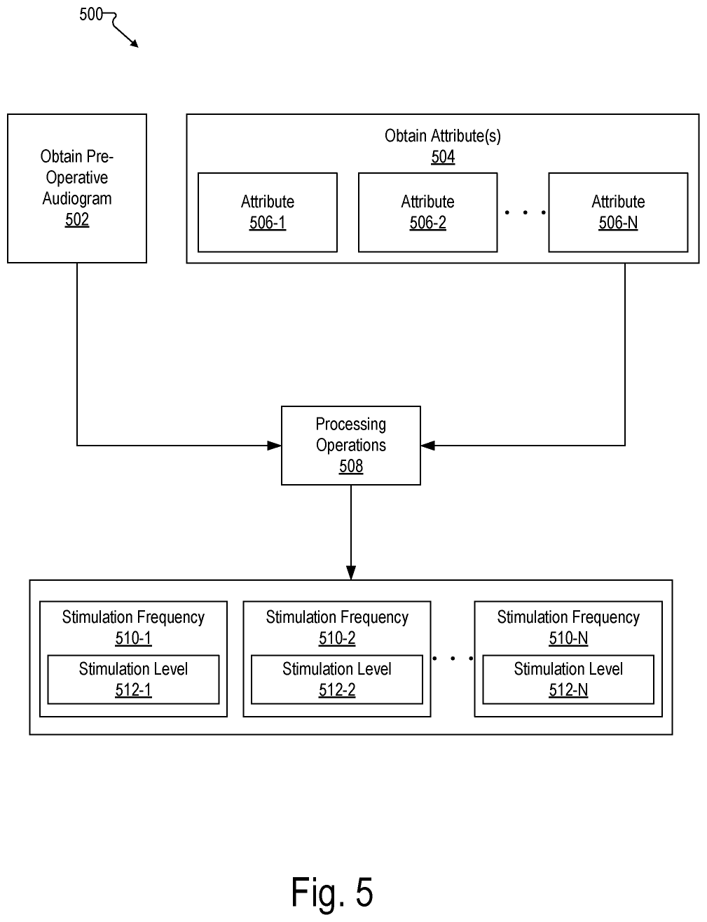

FIG. 5 illustrates an exemplary flow diagram 500 that depicts various operations that may be performed by system 400 to determine which stimulation frequencies to use. As shown in FIG. 5, system 400 may obtain a pre-operative audiogram of a recipient at operation 502. As used herein, an “pre-operative audiogram” may include any suitable representation of information that indicates a particular recipient's hearing capability across a range of frequencies. The pre-operative audiogram may be generated prior to a lead insertion procedure during which an electrode lead (e.g., electrode lead 104) having a plurality of electrodes is inserted into a cochlea of a recipient. System 400 may obtain the pre-operative audiogram in any suitable manner. For example, in certain implementations, system 400 may access the pre-operative audiogram from any suitable database. In certain alternative implementations, system 400 may generate or otherwise facilitate a user providing a user input to enter the pre-operative audiogram.

A pre-operative audiogram may be generated in any suitable manner. For example, prior to the lead insertion procedure, a hearing care professional may provide acoustic stimulus (e.g., beeps) at different frequencies to determine the hearing thresholds of the recipient across a range of frequencies. The pre-operative audiogram may then be generated based on responses provided by the recipient. Exemplary pre-operative audiograms are described herein.

At operation 504, system 400 may obtain one or more attributes 506 (e.g., attributes 506-1 through 506-N) associated with a lead insertion procedure. Attributes 506 may include any suitable attributes associated with a lead insertion procedure as may serve a particular implementation. For example, attributes 506 may include information indicative of a predicted insertion depth, information associated with additional cochlear implant patients, an intended insertion depth based on pre-operative imaging and/or system capabilities. System 400 may obtain attributes 506 in any suitable manner. For example, in certain examples, system 400 may determine a predicted insertion depth based on past lead insertion procedures performed on additional recipients, the anatomy (e.g., the size of the cochlea) of the recipient, an intended insertion depth based on CT imaging, and/or based on any other suitable information. In certain examples, the predicted insertion depth may be obtained based on a user input provided by a surgeon performing the lead insertion procedure. To illustrate an example, system 400 may receive a user input from a surgeon that selects an insertion depth of 300° based on the size of a particular recipient's cochlea and/or a desire to avoid going too deep within the cochlea, which may increase the chances of causing trauma to the cochlea.

The information associated with the additional cochlear implant patients may include any suitable information as may serve a particular implementation. For example, the information associated with the additional cochlear implant patients may include audiogram information, evoked response information, electrode lead path information, insertion depth information, and/or any other suitable information. The information associated with the additional cochlear implant patients may be obtained in any suitable manner. For example, the information associated with the additional cochlear implant patients may be obtained from any suitable database (e.g., a cloud database) that may store information associated with past insertion procedures. Such information may be collected and processed in any suitable manner by system 400 to facilitate improving outcomes of lead insertion procedures. For example, system 400 may use any suitable machine learning algorithm to process the information associated with past insertion procedures.

The system capabilities may include any suitable information that may indicate what system 400 is capable of performing. For example, the system capability information may include information indicating a maximum stimulation level that may be output by system 400 for each of a plurality of different stimulation frequencies. The system capability information may be obtained from any suitable location (e.g., memory 402).

At operation 508, system 400 may perform any suitable processing operations to process the pre-operative audiogram and/or one or more attributes such as those described herein. For example, at operation 506, system 400 may determine, based on the pre-operative audiogram and the one or more attributes, one or more stimulation frequencies for stimulation that is to be used during the lead insertion procedure to elicit evoked responses within the recipient. This may be accomplished in any suitable manner. For example, system 400 may determine, based on the pre-operative audiogram, that a recipient has good hearing at a first stimulation frequency and a second stimulation frequency that is different than the first stimulation frequency. Accordingly, system 400 may determine that the one or more stimulation frequencies should include the first stimulation frequency and the second stimulation frequency. To illustrate, the first stimulation frequency may correspond to 250 Hz and the second stimulation frequency may correspond to 500 Hz.

In certain examples, having stimulation frequencies that are multiples of one another may result in peaks occurring at other frequencies. For example, a stimulation frequency of 250 Hz may show up as a peak for a stimulation frequency of 500 Hz. To avoid this, system 400 may select nonharmonic stimulation frequencies to use in certain examples during a lead insertion procedure. To illustrate, in the example described above, instead of selecting 250 Hz for the first frequency and 500 Hz for the second frequency, system 400 may select 245 Hz and 510 Hz to use during the lead insertion procedure.

In certain examples, system 400 may be further configured to determine, based on the one or more attributes, a stimulation level to use during the lead insertion procedure for each of the one or more stimulation frequencies. This may be accomplished in any suitable manner. For example, system 400 may determine, based on a recipient's hearing capability and the maximum stimulation levels that may be output by system 400, that a first stimulation frequency should be output at a first stimulation level, a second stimulation frequency should be output at a second stimulation level, and that a third stimulation frequency should be output at a third stimulation level. In certain examples, system 400 may determine which stimulation level to use for a given stimulation frequency for a particular recipient based on statistical information associated with past lead insertion procedures performed with respect to additional recipients. For example, system 400 may access statistical information of past recipients that have similar pre-operative audiograms, similar anatomy, and/or were subject to similar stimulation levels that resulted in favorable outcomes during lead insertion procedures. Based on such information, system 400 may identify the optimal stimulation levels to use during the lead insertion procedure for the particular recipient.

As shown in FIG. 5, the output for operation 506 may include a plurality of stimulation frequencies 510 (e.g., stimulation frequencies 510-1 through 510-N) and a plurality of stimulation levels 512 (e.g., stimulation levels 512-1 through 512-N). In certain examples, plurality of stimulation frequencies 510 may be concurrently used during an insertion procedure to elicit evoked responses with a recipient. For example, stimulation at stimulation frequence 510-1 and stimulation level 512-1 may be applied during a time period during the lead insertion procedure and stimulation at stimulation frequency 510-2 and stimulation level 512-2 may be concurrently applied to the recipient during the time period.

In certain alternative implementations, each stimulation frequency 510 may be used at a different time during the lead insertion procedure. For example, stimulation at stimulation frequency 510-1 and stimulation level 512-1 may be applied during a first time period, then stimulation at stimulation frequency 510-2 and stimulation level 512-2 may be applied during a second time period that does not overlap the first time period.

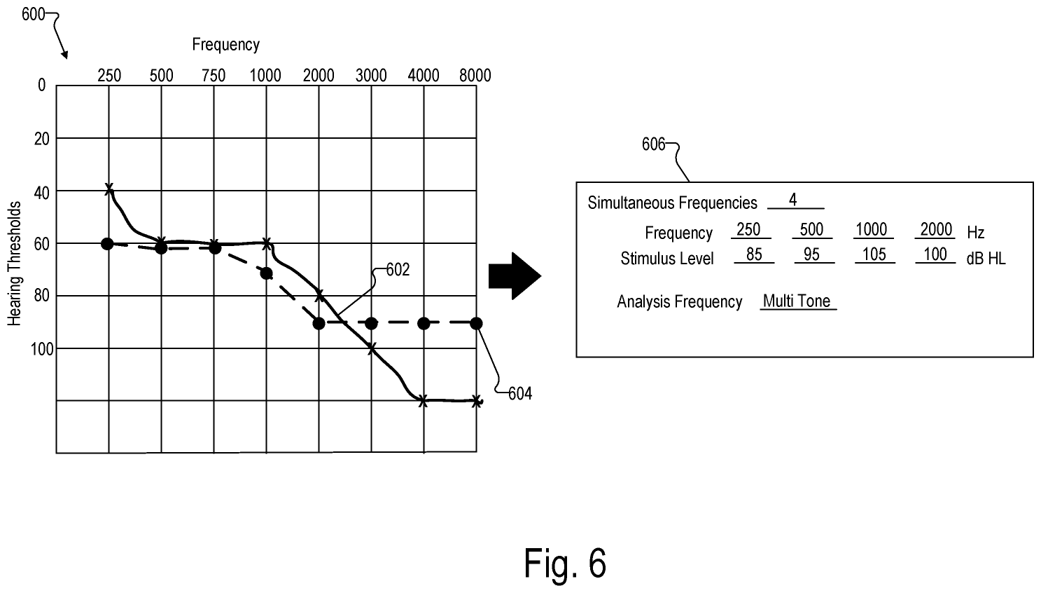

FIGS. 6-8 depict exemplary pre-operative audiograms that may be obtained and used by system 400 to facilitate identifying one or more stimulus frequencies to use during a lead insertion procedure. As shown in FIG. 6, pre-operative audiogram 600 includes a first curve 602 representing hearing thresholds for a first ear of a recipient across a range of frequencies and a second curve 604 representing hearing thresholds for a second ear of the recipient across the range of frequencies. The recipient may be scheduled to receive a cochlear implant in the cochlea of the second ear. As such, system 400 may select which frequencies to use, at least in part, based on second curve 604. Interface 606 depicts parameters that may be selected by system 400 based on pre-operative audiogram 600 and one or more other attributes, such as those described herein. In the example shown in FIG. 6, four frequencies (250, 500, 1000, and 2000 Hz) have been selected by system 400 for simultaneous multi-tone presentation during the lead insertion procedure. In addition, a respective stimulus level is indicated for each of the stimulus frequencies. In the particular example shown in FIG. 6, a stimulus level of 85 dB has been set for the stimulus frequency 250 Hz, a stimulus level of 95 dB has been set for stimulus frequency 500 Hz, a stimulus level of 105 dB has been set for stimulus frequency 1000 Hz, and a stimulus level of 100 dB has been set for frequency 2000 Hz. The four stimulus frequencies depicted in interface 606 are provided for illustrative purposes only. It is understood that in certain examples slightly different stimulus frequencies may be selected than those shown in FIG. 6 to avoid having stimulation frequencies that are harmonic multiples of each other. For example, 995 Hz and 1010 Hz may be selected as alternative stimulation frequencies for the third and fourth stimulation frequencies in certain examples.

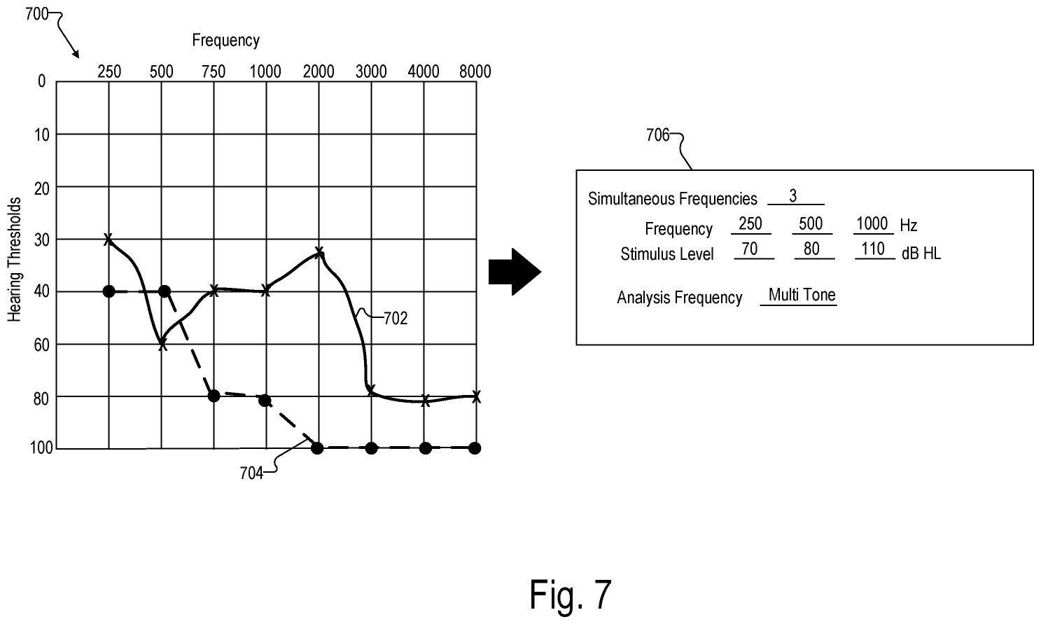

FIG. 7 shows an example where only three stimulus frequencies have been selected. As shown in FIG. 7, pre-operative audiogram 700 includes a first curve 702 representing hearing thresholds for a first ear of a recipient across a range of frequencies and a second curve 704 representing hearing thresholds for a second ear of the recipient across the range of frequencies. The recipient associated with pre-operative audiogram 700 may be scheduled to receive a cochlear implant in the cochlea of the first ear. As such, system 400 may select which frequencies to use based, at least in part, on second curve 704. Interface 706 depicts parameters that may be selected by system 400 based on pre-operative audiogram 700 and one or more other attributes, such as those described herein. In the example shown in FIG. 7, three frequencies (250, 500, and 1000 Hz) have been selected for simultaneous multi-tone presentation during the lead insertion procedure. In addition, a respective stimulus level is indicated for each of the stimulus frequencies. For example, a stimulus level of 70 dB has been set for the stimulus frequency 250 Hz.

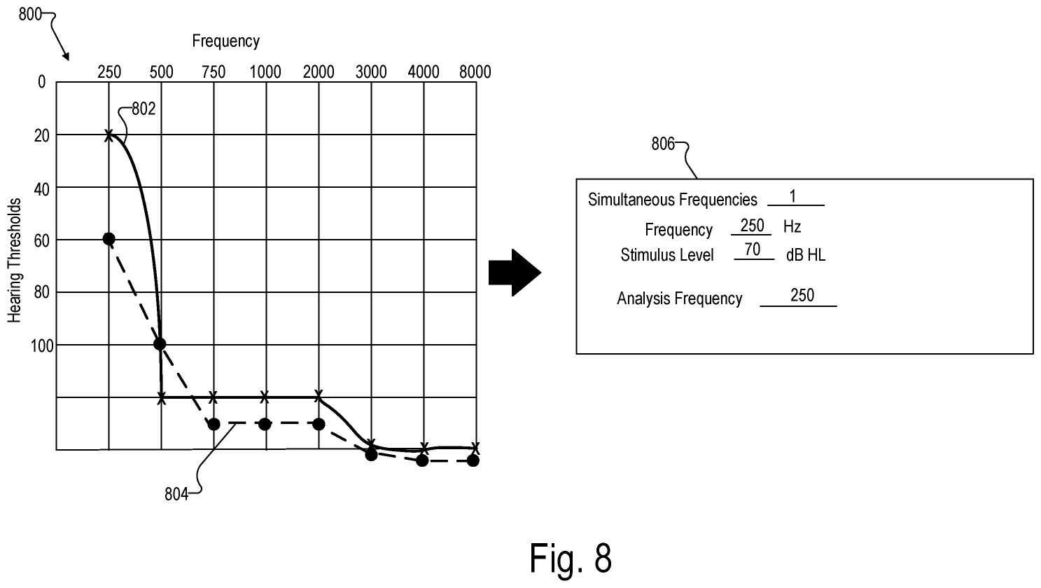

FIG. 8 shows an example where only one stimulus frequencies has been selected. As shown in FIG. 8, pre-operative audiogram 800 includes a first curve 802 representing hearing thresholds for a first ear of a recipient across a range of frequencies and a second curve 804 representing hearing thresholds for a second ear of the recipient across the range of frequencies. The recipient associated with pre-operative audiogram 800 may be scheduled to receive a cochlear implant in the cochlea of the first ear. However, that particular recipient may only have hearing capability with respect to a stimulus frequency of 250 Hz in the first ear. As such, system 400 may select a stimulus frequency of 250 Hz to use during the lead insertion procedure. Interface 806 depicts parameters that may be selected by system 400 based on pre-operative audiogram 800 and one or more other attributes, such as those described herein. In the example shown in FIG. 8, a stimulus level of 70 dB has been set for the stimulus frequency 250 Hz.

In certain implementations, system 400 may monitor, during a lead insertion procedure, the evoked responses elicited within the recipient based on the one or more stimulation frequencies. Based on the monitoring of the evoked responses, system 400 may perform any suitable process that may facilitate the lead insertion procedure. In certain examples, system 400 may determine, based on the monitoring of the evoked responses, that trauma has occurred within the cochlea during the lead insertion procedure. This may be accomplished in any suitable manner. For example, system 400 may detect a decrease in the amplitude of one or more evoked responses and determine, based on the decrease, that an insertion trauma (e.g., pushing against the basiliar membrane, translocation, etc.) has occurred.

In certain examples, system 400 may provide feedback regarding the insertion condition during the lead insertion procedure based on the monitoring of the evoked responses. The feedback may be provided in any suitable manner as may serve a particular implementation. For example, the feedback may include an audible sound notification, a graphical notification, and/or any other suitable notification.

To illustrate an example, system 400 may present, based on the insertion condition, a notification that a translocation event is about to occur or has occurred during the lead insertion procedure. System 400 may present the notification in any suitable manner. For example, system 400 may display the notification by way of a display device (e.g., within a graphical user interface displayed by the display device). To illustrate, system 400 may display the notification by way of a display device within a microscope used by a surgeon to perform the lead insertion procedure. In some examples, the display device may be included in an augmented reality system that produces a predicted image of the electrode lead's current status based on the insertion condition. Additionally or alternatively, system 400 may present an audible sound representative of the notification.

Additionally or alternatively, system 400 may be configured to provide, based on the insertion condition, one or more instructions regarding how to correct and/or prevent the translocation event. For example, system 400 may provide (e.g., display on a display device) one or more steps that may be performed to at least partially retract the electrode lead from the cochlea and then correctly reinsert the electrode lead into the cochlea.

Additionally or alternatively, system 400 may be configured to stop (e.g., automatically) the lead insertion procedure based on the insertion condition. This may be performed in any suitable manner. For example, if a computer-assisted lead insertion system (e.g., a robotic lead insertion system) is being used to insert the electrode lead, system 400 may direct the computer-assisted lead insertion system to stop the lead insertion procedure (e.g., by transmitting one or more commands to the computer-assisted lead insertion system). System 400 may be further configured to transmit one or more additional commands to the computer-assisted lead insertion system to cause the electrode lead to be retracted until the insertion condition indicates that the insertion trauma/translocation event is no longer occurring or that the insertion trauma/translocation event is no longer about to occur.

In some examples, system 400 may use a machine learning model to perform any of the operations described herein. For example, system 400 may use a machine learning model to assist in processing statistical information associated with lead insertion procedures performed with respect to additional cochlear implant recipients.

Although the preceding disclosure describes identifying stimulus frequencies for electrode leads configured for use with a cochlear implant system (e.g., cochlear implant system 100), it is understood that concepts such as those described herein may be applied to any other suitable type of implantable electrode or externally used electrode that may be implemented in any other suitable context. For example, concepts such as those described herein may be applied to electrode leads used in neuroprosthetic systems, neurostimulation systems, optical nerve stimulation systems, cardiac stimulation systems, etc.

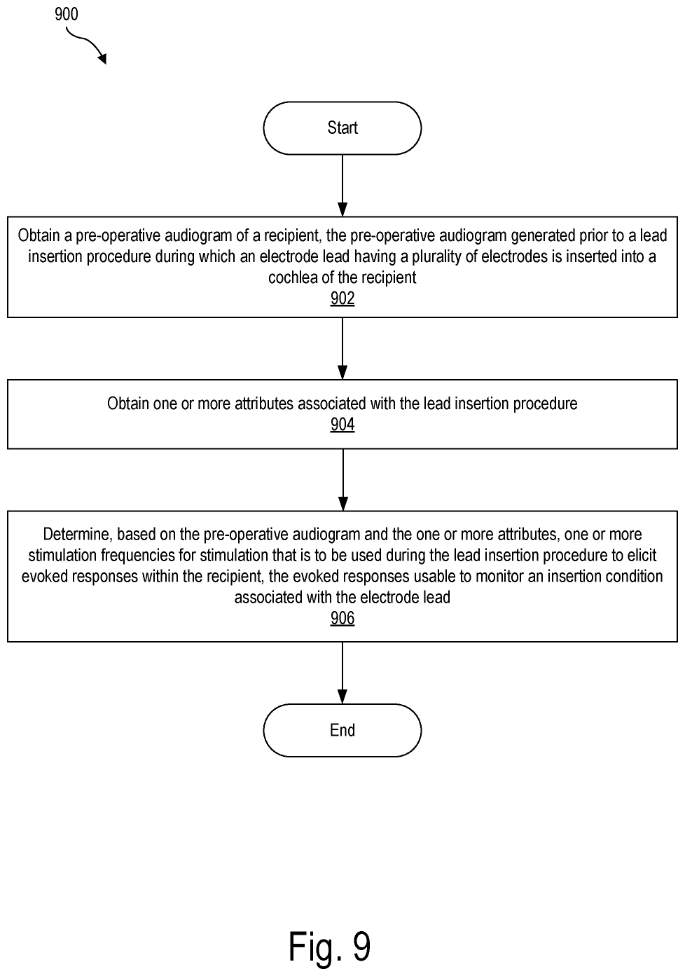

FIG. 9 illustrates an exemplary method 900 for identifying stimulus frequencies to use during a lead insertion procedure. While FIG. 9 illustrates exemplary operations according to one embodiment, other embodiments may omit, add to, reorder, and/or modify any of the operations shown in FIG. 9.

At operation 902, a lead insertion management system (e.g., lead insertion management system 400) may obtain a pre-operative audiogram of a recipient. The pre-operative audiogram may be generated prior to a lead insertion procedure during which an electrode lead having a plurality of electrodes is inserted into a cochlea of the recipient. Operation 902 may be performed in any of the ways described herein.

At operation 904, the lead insertion management system may obtain one or more attributes associated with the lead insertion procedure. Operation 904 may be performed in any of the ways described herein.

At operation 906, the lead insertion management system may determine, based on the pre-operative audiogram and the one or more attributes, one or more stimulation frequencies for stimulation (e.g., acoustic stimulation) that is to be used during the lead insertion procedure to elicit evoked responses within the recipient. The evoked responses may be usable to monitor an insertion condition associated with the electrode lead. Operation 906 may be performed in any of the ways described herein.

In some examples, a computer program product embodied in a non-transitory computer-readable storage medium may be provided. In such examples, the non-transitory computer-readable storage medium may store computer-readable instructions in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g., a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

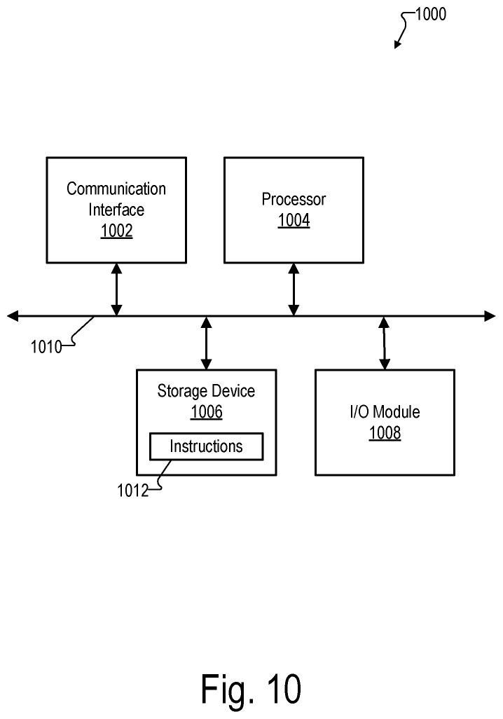

FIG. 10 illustrates an exemplary computing device 1000 that may be specifically configured to perform one or more of the processes described herein. As shown in FIG. 10, computing device 1000 may include a communication interface 1002, a processor 1004, a storage device 1006, and an input/output (“I/O”) module 1008 communicatively connected one to another via a communication infrastructure 1010. While an exemplary computing device 1000 is shown in FIG. 10, the components illustrated in FIG. 10 are not intended to be limiting. Additional or alternative components may be used in other embodiments. Components of computing device 1000 shown in FIG. 10 will now be described in additional detail.

Communication interface 1002 may be configured to communicate with one or more computing devices. Examples of communication interface 1002 include, without limitation, a wired network interface (such as a network interface card), a wireless network interface (such as a wireless network interface card), a modem, an audio/video connection, and any other suitable interface.

Processor 1004 generally represents any type or form of processing unit capable of processing data and/or interpreting, executing, and/or directing execution of one or more of the instructions, processes, and/or operations described herein. Processor 1004 may perform operations by executing computer-executable instructions 1012 (e.g., an application, software, code, and/or other executable data instance) stored in storage device 1006.

Storage device 1006 may include one or more data storage media, devices, or configurations and may employ any type, form, and combination of data storage media and/or device. For example, storage device 1006 may include, but is not limited to, any combination of the non-volatile media and/or volatile media described herein. Electronic data, including data described herein, may be temporarily and/or permanently stored in storage device 1006. For example, data representative of computer-executable instructions 1012 configured to direct processor 1004 to perform any of the operations described herein may be stored within storage device 1006. In some examples, data may be arranged in one or more databases residing within storage device 1006.

I/O module 1008 may include one or more I/O modules configured to receive user input and provide user output. I/O module 1008 may include any hardware, firmware, software, or combination thereof supportive of input and output capabilities. For example, I/O module 1008 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1008 is configured to provide graphical data to a display for presentation to a user. The graphical data may be representative of one or more graphical user interfaces and/or any other graphical content as may serve a particular implementation.

In some examples, any of the systems, hearing devices, computing devices, and/or other components described herein may be implemented by computing device 1000. For example, memory 402 may be implemented by storage device 1006, and processor 404 may be implemented by processor 1004.

Additional advantages and features of the present disclosure can be further described by the following statements:

1. A system comprising: a memory storing instructions; and a processor communicatively coupled to the memory and configured to execute the instructions to perform a process comprising: obtaining a pre-operative audiogram of a recipient, the pre-operative audiogram generated prior to a lead insertion procedure during which an electrode lead having a plurality of electrodes is inserted into a cochlea of the recipient; obtaining one or more attributes associated with the lead insertion procedure; and determining, based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

2. The system of the preceding statement, wherein the process further comprises determining, based on the one or more attributes, a stimulation level to use during the lead insertion procedure for each of the one or more stimulation frequencies.

3. The system of any of the preceding statements, wherein the one or more stimulation frequencies include a plurality of stimulation frequencies that are concurrently used during the insertion procedure to elicit the evoked responses within the recipient.

4. The system of any of the preceding statements, wherein: the one or more stimulation frequencies include a first stimulation frequency and a second stimulation frequency that is different than the first stimulation frequency; and the first stimulation frequency is not harmonic with respect to the second stimulation frequency.

5. The system of any of the preceding statements, wherein the process further comprises: monitoring, during the lead insertion procedure, the evoked responses elicited within the recipient based on the one or more stimulation frequencies; and providing, based on the monitoring of the evoked responses, feedback regarding the insertion condition during the lead insertion procedure.

6. The system any of the preceding statements, wherein the feedback includes one or more of an audible sound notification or a graphical notification presented for display by way of a display device.

7. The system any of the preceding statements, wherein the process further comprises: monitoring, during the lead insertion procedure, the evoked responses elicited within the recipient based on the one or more stimulation frequencies; and determining, based on the monitoring of the evoked responses, that trauma has occurred within the cochlea during the lead insertion procedure.

8. The system of any of the preceding statements, wherein the one or more attributes associated with the lead insertion procedure include one or more of an intended insertion depth based on anatomy or pre-operative imaging.

9. The system any of the preceding statements, wherein the one or more attributes associated with the lead insertion procedure include one or more of a predicted insertion depth, statistical information associated with additional cochlear implant recipients, or system capabilities.

10. A computer program product embodied on a non-transitory computer readable medium and comprising computer instructions for: obtaining a pre-operative audiogram of a recipient, the pre-operative audiogram generated prior to a lead insertion procedure during which an electrode lead having a plurality of electrodes is inserted into a cochlea of the recipient; obtaining one or more attributes associated with the lead insertion procedure; and determining, based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

11. The computer program product of the preceding statement, wherein the computer instructions are further for determining, based on the one or more attributes, a stimulation level to use during the lead insertion procedure for each of the one or more stimulation frequencies.

12. The computer program product of any of the preceding statements, wherein the one or more stimulation frequencies include a plurality of stimulation frequencies that are concurrently used during the insertion procedure to elicit the evoked responses within the recipient.

13. The computer program product of any of the preceding statements, wherein: the one or more stimulation frequencies include a first stimulation frequency and a second stimulation frequency that is different than the first stimulation frequency; and the first stimulation frequency is not harmonic with respect to the second stimulation frequency.

14. The computer program product of any of the preceding statements, wherein the one or more attributes associated with the lead insertion procedure include one or more of an intended insertion depth based on anatomy or pre-operative imaging.

15. The computer program product of any of the preceding statements, wherein the one or more attributes associated with the lead insertion procedure include one or more of a predicted insertion depth, statistical information associated with additional cochlear implant recipients, or system capabilities.

16. The computer program product of any of the preceding statements, wherein the instructions are further for: monitoring, during the lead insertion procedure, the evoked responses elicited within the recipient based on the one or more stimulation frequencies; and providing, based on the monitoring of the evoked responses, feedback regarding the insertion condition during the lead insertion procedure.

17. A method comprising: obtaining, by a lead insertion management system, a pre-operative audiogram of a recipient, the pre-operative audiogram generated prior to a lead insertion procedure during which an electrode lead having a plurality of electrodes is inserted into a cochlea of the recipient; obtaining, by the lead insertion management system, one or more attributes associated with the lead insertion procedure; and determining, by the lead insertion management system and based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

18. The method of the preceding statement, further comprising determining, by the lead insertion management system and based on the one or more attributes, a stimulation level to use during the lead insertion procedure for each of the one or more stimulation frequencies.

19. The method of any of the preceding statements, further comprising: monitoring, by the lead insertion management system and during the lead insertion procedure, the evoked responses elicited within the recipient based on the one or more stimulation frequencies; and providing, by the lead insertion management system and based on the monitoring of the evoked responses, feedback regarding the insertion condition during the lead insertion procedure.

20. The method of any of the preceding statements, further comprising: monitoring, by the lead insertion management system and during the lead insertion procedure, the evoked responses elicited within the recipient based on the one or more stimulation frequencies; and determining, by the lead insertion management system and based on the monitoring of the evoked responses, that trauma has occurred within the cochlea during the lead insertion procedure.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a memory storing instructions; and
a processor communicatively coupled to the memory and configured to execute the instructions to perform a process comprising:
obtaining a pre-operative audiogram of a recipient, the pre-operative audiogram generated prior to a lead insertion procedure during which an electrode lead having a plurality of electrodes is inserted into a cochlea of the recipient;
obtaining one or more attributes associated with the lead insertion procedure; and
determining, based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

2. The system of claim 1, wherein the process further comprises determining, based on the one or more attributes, a stimulation level to use during the lead insertion procedure for each of the one or more stimulation frequencies.

3. The system of claim 1, wherein the one or more stimulation frequencies include a plurality of stimulation frequencies that are concurrently used during the insertion procedure to elicit the evoked responses within the recipient.

4. The system of claim 1, wherein:
the one or more stimulation frequencies include a first stimulation frequency and a second stimulation frequency that is different than the first stimulation frequency; and
the first stimulation frequency is not harmonic with respect to the second stimulation frequency.

5. The system of claim 1, wherein the process further comprises:
monitoring, during the lead insertion procedure, the evoked responses elicited within the recipient based on the one or more stimulation frequencies; and
providing, based on the monitoring of the evoked responses, feedback regarding the insertion condition during the lead insertion procedure.

6. The system of claim 5, wherein the feedback includes one or more of an audible sound notification or a graphical notification presented for display by way of a display device.

7. The system of claim 1, wherein the process further comprises:
monitoring, during the lead insertion procedure, the evoked responses elicited within the recipient based on the one or more stimulation frequencies; and
determining, based on the monitoring of the evoked responses, that trauma has occurred within the cochlea during the lead insertion procedure.

8. The system of claim 1, wherein the one or more attributes associated with the lead insertion procedure include one or more of an intended insertion depth based on anatomy or pre-operative imaging.

9. The system of claim 1, wherein the one or more attributes associated with the lead insertion procedure include one or more of a predicted insertion depth, statistical information associated with additional cochlear implant recipients, or system capabilities.

10. A computer program product embodied on a non-transitory computer readable medium and comprising computer instructions for:
obtaining a pre-operative audiogram of a recipient, the pre-operative audiogram generated prior to a lead insertion procedure during which an electrode lead having a plurality of electrodes is inserted into a cochlea of the recipient;
obtaining one or more attributes associated with the lead insertion procedure; and
determining, based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

11. The computer program product of claim 10, wherein the computer instructions are further for determining, based on the one or more attributes, a stimulation level to use during the lead insertion procedure for each of the one or more stimulation frequencies.

12. The computer program product of claim 10, wherein the one or more stimulation frequencies include a plurality of stimulation frequencies that are concurrently used during the insertion procedure to elicit the evoked responses within the recipient.

13. The computer program product of claim 10, wherein:
the one or more stimulation frequencies include a first stimulation frequency and a second stimulation frequency that is different than the first stimulation frequency; and
the first stimulation frequency is not harmonic with respect to the second stimulation frequency.

14. The computer program product of claim 10, wherein the one or more attributes associated with the lead insertion procedure include one or more of an intended insertion depth based on anatomy or pre-operative imaging.

15. The computer program product of claim 10, wherein the one or more attributes associated with the lead insertion procedure include one or more of a predicted insertion depth, statistical information associated with additional cochlear implant recipients, or system capabilities.

16. The computer program product of claim 10, wherein the instructions are further for:
monitoring, during the lead insertion procedure, the evoked responses elicited within the recipient based on the one or more stimulation frequencies; and
providing, based on the monitoring of the evoked responses, feedback regarding the insertion condition during the lead insertion procedure.

17. A method comprising:
obtaining, by a lead insertion management system, a pre-operative audiogram of a recipient, the pre-operative audiogram generated prior to a lead insertion procedure during which an electrode lead having a plurality of electrodes is inserted into a cochlea of the recipient;
obtaining, by the lead insertion management system, one or more attributes associated with the lead insertion procedure; and
determining, by the lead insertion management system and based on the pre-operative audiogram and the one or more attributes, one or more stimulation frequencies for stimulation that is to be used during the lead insertion procedure to elicit evoked responses within the recipient, the evoked responses usable to monitor an insertion condition associated with the electrode lead.

18. The method of claim 17, further comprising determining, by the lead insertion management system and based on the one or more attributes, a stimulation level to use during the lead insertion procedure for each of the one or more stimulation frequencies.

19. The method of claim 17, further comprising:
monitoring, by the lead insertion management system and during the lead insertion procedure, the evoked responses elicited within the recipient based on the one or more stimulation frequencies; and
providing, by the lead insertion management system and based on the monitoring of the evoked responses, feedback regarding the insertion condition during the lead insertion procedure.

20. The method of claim 17, further comprising:
monitoring, by the lead insertion management system and during the lead insertion procedure, the evoked responses elicited within the recipient based on the one or more stimulation frequencies; and
determining, by the lead insertion management system and based on the monitoring of the evoked responses, that trauma has occurred within the cochlea during the lead insertion procedure.
